(19) United States
(12) Patent Application Publication
(10) Pub. No.: US 2002/0111496 A1
(43) Pub. Date: Aug. 15, 2002

(54) Inhibitors of glycosyltransferase enzymes

(76) Inventors: Benjamin A. Horenstein, Gainesville, FL (US); Hongbin Sun, Gainesville, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/067,495

(22) Filed: Feb. 4, 2002

Related U.S. Application Data
(60) Provisional application No. 60/266,128, filed on Feb. 2, 2001.

(51) Int. Cl. C07D 293/00; C07D 285/14; C07D 513/00

(52) U.S. Cl. 548/100; 548/122; 548/126

(57) ABSTRACT

The subject invention provides compounds and methods of producing compounds, which are useful inhibitors of glycosyltransferase enzymes. These compounds represent a new class of glycosyltransferase inhibitors and are potent inhibitors of sialyltransferase. The subject invention also provides methods of treating diseases or conditions associated with glycosytransferases. Methods of modulating the activity of glycosytransferases are also provided.

### CROSS-REFERENCE TO RELATED APPLICATION

The present application claims priority to U.S. Provisional Patent Application Serial No. 60/266,128, filed Feb. 2, 2001, which is hereby incorporated by reference herein in its entirety, including any figures, tables, or drawings.

The subject invention was made with government support under a research project supported by the National Science Foundation, Contract Number MCB 950-1886. The government may have certain rights in this invention.

### BACKGROUND OF THE INVENTION

Sialic acids play an important role in a variety of biological processes. (Rosenberg, A. [1995] Biology of Sialic Acids. Plenum, New York; Reutter, W.; Stasche, R.; Stehling, P.; Baum, O. [1997] Glycosciences (Eds.: Gabius, H. J., Gabius, S.), Chapman & Hall, Weinheim, pp. 245-259.) They are usually attached to the terminal positions of glycoproteins, glycolipids and oligosaccharides. Of more than 100 different sialic acids, N-acetylneuraminic acid (NeuAc) is the most abundant one. (Gottschalk, A. [1951] Nature 167, 845.) The sugar-nucleotide CMP-NeuAc 1 (FIG. 1) is the key substrate for biosynthesis of sialylated glycoconjugates in which CMP-NeuAc is transferred by sialyltransferases to an acceptor hydroxyl group in a variety of substrates including polysialic acids, glycoproteins and gangliosides. (Harduin-Lepers, A. Recchi, M. A.; Delannoy, P. [1995] Glycobiology 5, 741; Varki, A. [1992] Glycobiology 2, 25; Reglero, A.; Rodriguez-Aparicio, L. B.; Lueng, J. M. [1993] Int. J. Biochem. 11, 1517; Hayes, B. K.; Varki, A. [1993] J. Biol. Chem. 268, 16155.)

These glycosylation patterns constitute important binding determinants for various cell-cell interactions which include masking of trypanosomal immunogenicity, viral infection and replication, and cell adhesion. (Lowe, J. B. [1994] Molecular Glycobiology; Fucuda, M.; Hindsgaul, O., Eds.; Oxford University Press: New York, pp 163-205; Powell, L. D.; Varki, A. [1995] J. Biol. Chem. 270, 14243; Crocker, P. R.; Kelm, S.; Hartell, A.; Freeman, S.; Nath, D.; Vinson, M.; Mucklow, S. [1996] Biochem. Soc. Trans. 24, 150.) Therefore, development of potent and selective inhibitors of sialyltransferases may be useful in a variety of biochemical applications. However, only a few potent inhibitors of sialyltransferases have been developed. (Schaub, C.; Muller, B.; Schmidt, R. R. [1998] Glycoconjugate J. 15, 345; Muller, B.; Schaub, C.; Schimdt, R. R. [1998] Angew. Chem. Int. Ed., 37, 2893; Amann, F.; Schaub, C.; Muller, B.; Schmidt, R. R. [1998] Chem. Eur. J. 4, 1106.)

### SUMMARY OF THE INVENTION

The subject invention provides compounds, and methods of producing compounds, which are useful inhibitors of glycosyltransferase enzymes. These compounds represent a new class of glycosyltransferase inhibitors and are potent inhibitors of sialyltransferases. The subject invention also provides methods of treating diseases or conditions associated with glycosyltransferases. Methods of modulating the activity of glycosyltransferases are also provided.

### BRIEF DESCRIPTION OF THE FIGURES

FIG. 1 provides the structure of the sugar nucleotide CMP-NeuAc.

FIG. 2 depicts the structure of compounds according to the instant invention.

FIG. 3 illustrates the oxocarbenium transition state of the sugar nucleotide CMP-NeuAc.

FIGS. 4A and 4B provide exemplary compounds of the instant invention containing cytosine.

FIG. 5 provides an exemplary reaction scheme for the production of compounds according to the instant invention. Reagents and conditions: a) i) m-CPBA; ii) NaBH₄, 48% for 2 steps. b) TBDMSC, or TBDPSC₁, imidazole. c) i) BH₃.SMe₂; ii) H₂O₂, NaOH, 90-95%. d) CrO₃, pyridine. e) Tf₂O, 2,6-di-tert-butyl-4-methylpyridine. f) Pd(OaC)₂, PPh₃, Et₃N, MeOH, CO, DMF, r.t., 80%. g) TBAF, THF, 85%.

FIG. 6 provides an alternative reaction scheme for the production of compounds according to the instant invention. Reagents and conditions: a) i) DMTrCl, pyridine; ii) Ac₂O, pyridine; iii) 90% AcOH, 41% overall yield. b) i) 11, tetrazole, CH₂Cl2; ii) t-BuOOH. iii) Et₃N, CH₂Cl2, 51% for 3 steps. c) i) NaOMe, MeOH—H₂O; ii) HPLC (NH₄HCO₃—MeOH—H₂O); iii) Amberlite IR-120, 30%.

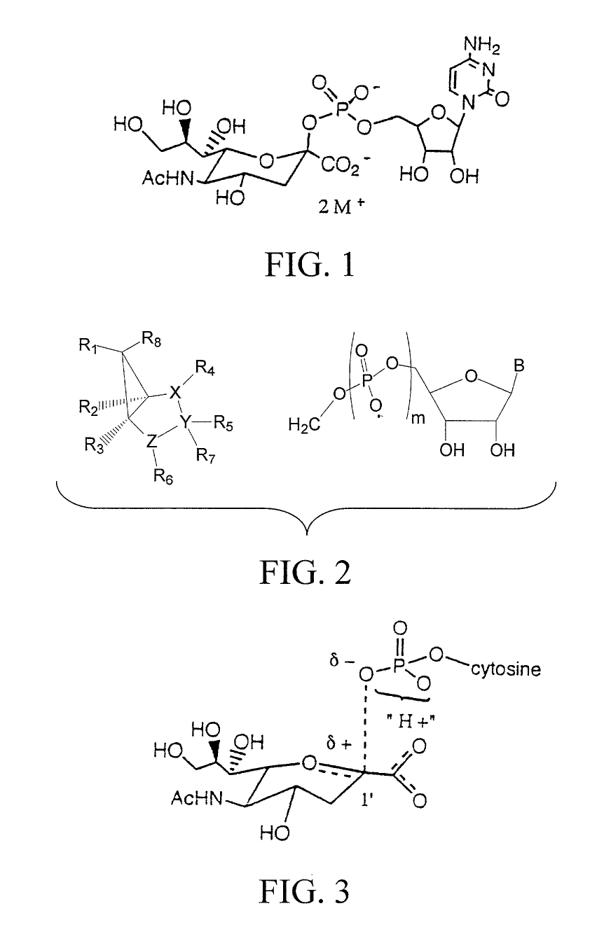

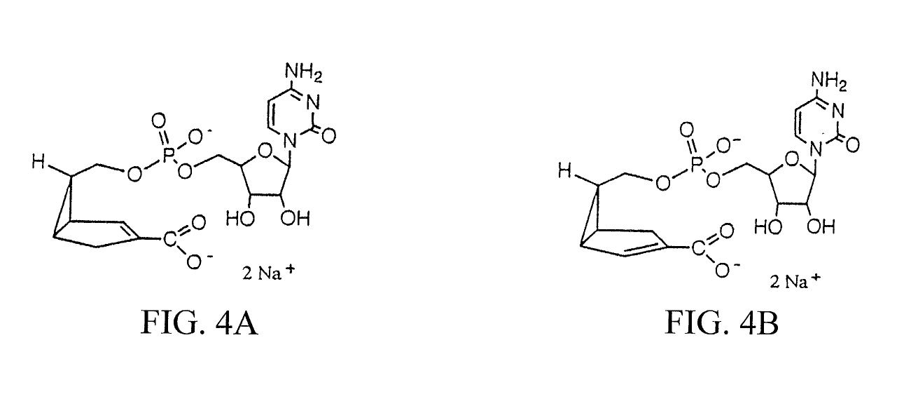

### DETAILED DESCRIPTION OF THE INVENTION

The subject invention provides compounds and methods of producing said compounds, which are useful inhibitors of glycosyltransferase enzymes. These compounds represent a new class of glycosyltransferase inhibitors. In one embodiment, the inhibitors are potent inhibitors of sialyltransferases. In a more preferred embodiment, the inhibitors exhibit significant inhibition of both 2,3 and 2,6-sialytransferases.

The subject invention provides compounds of the formula:

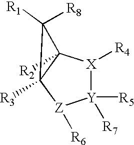

wherein R₁-R₈ are moieties selected from the group consisting of R₉, CH₃, alkyl groups, substituted alkyl groups, halogen, carboxyl, hydroxyl, phosphate, phosphonate, sugar residues, sugars, aryl, nucleosides, nucleoside monophosphates, nucleoside disphosphates, nucleoside triphosphates, and hydrogen;

R₉ is

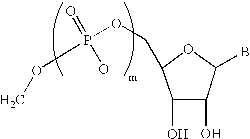

wherein B is adenine, thymine, guanine, cytosine, uracil, nicotinamide, or analogs thereof; and

m is 1 or 2;

X, Y, and Z are carbon, nitrogen, oxygen, or sulfur; and

a double bond may, optionally, exist between atoms X and Y or atoms Y and Z.

Substituted alkyl groups can be substituted at any available position with a moiety selected from the group consisting of C₁₋₆ alkyl, halogen, CN, OH, COOH, NO₂, NH₂, SO₂₋₄, C₁₋₂₀ heteroalkyl, C₂₋₂₀ alkenyl, alkynyl, akynyl-aryl, alkynyl-heteroaryl, aryl, C₁₋₂₀ alkyl-aryl, C₂₋₂₀ alkenyl-aryl, heteroaryl, C₁₋₂₀ alkyl-heteroaryl, C₂₋₂₀ alkenyl-heteroaryl, cycloalkyl, heterocycloalkyl, C₁₋₂₀ alkyl-heteroycloalkyl, and C₁₋₂₀ alkyl-cycloalkyl, any of which may be, optionally, substituted with a moiety selected from the group consisting of C₁₋₆ alkyl, halogen, OH, NH₂, CN, NO₂, COOH, or SO₂₋₄. Exemplary heterocyclic groups include, but not limited to, morpholine, triazole, imidazole, pyrrolidine, piperidine, piperazine, pyrrole, dihydropyridine, aziridine, thiazolidine, thiazoline, thiadiazolidine, or thiadiazoline. The value for n may be from 1 to 19. In other embodiments, the alkyl groups can be C₁-C₆ alkyl groups.

Novel compounds according to the invention can be provided in their salt form. Thus, the invention includes pharmaceutically acceptable salts, for example acid addition salts derived from inorganic or organic acids, such as hydrohlorides, hydrobromides, p-toluenesulfonates, phosphates, sulfates, perchlorates, acetates, trifluororacetates, propionates, citrates, malonates, succinates, lactates, oxalates, tartrates, and benzoates. Salts may also be derived from bases (organic and inorganic), such as alkali metal salts (e.g., magnesium or calcium salts), or organic amine salts, such as morpholine, piperidine, dimethylamine, or diethylamine salts.

Also provided by the subject invention are compositions comprising the compounds of the subject invention and a carrier. In one embodiment, the compositions contain all diastereoisomers arising from the synthesis of the compounds of the invention. In another embodiment, the diastereoisomers are separated from one another and compositions according to the invention contain each respective isolated diastereoisomer. In another embodiment, compositions containing the isolated diastereoisomers may be combined into a mixture containing two or more of the isolated diastereoisomer compositions. Methods of resolving diastereoisomers are well known in the art.

In another embodiment (when R is H or CH₂), the subject invention provides for racemic mixtures and isolated enantiomeric compounds of the “scorpio” structure. Thus, the invention also provides for compositions containing the “scorpio” racemate and compositions containing the isolated enantiomeric forms of the “scorpio” structure. The various enantiomeric forms of the compounds may be isolated according to methods well known to the skilled artisan.

Furthermore, the compounds of the invention may be administered in conjunction with other compounds or compositions thereof. These compounds and compositions may include antibiotics, antiviral agents, chemotherapeutic agents, or immunosuppressant agents.

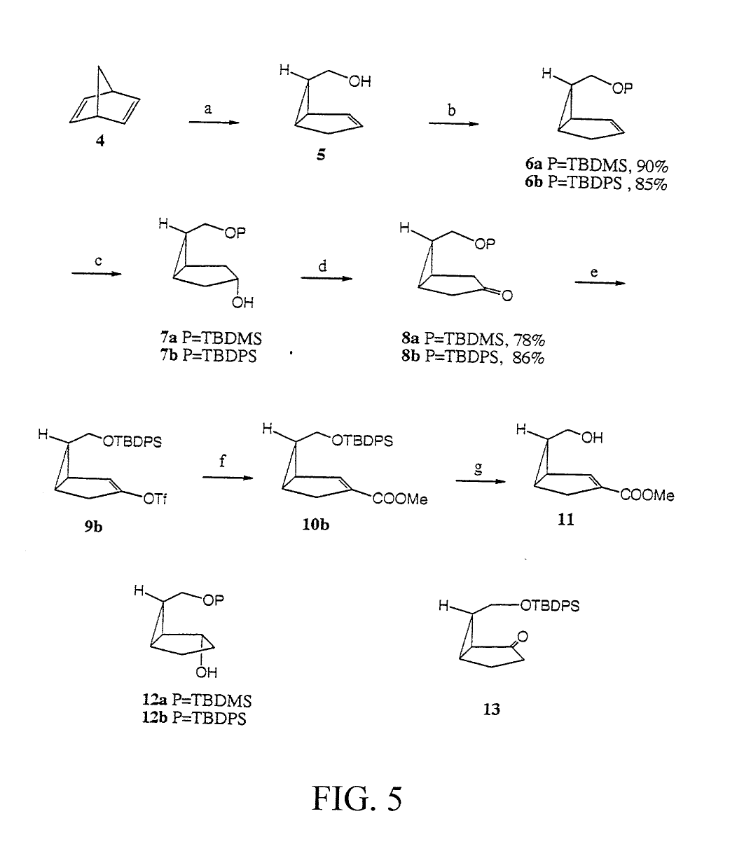

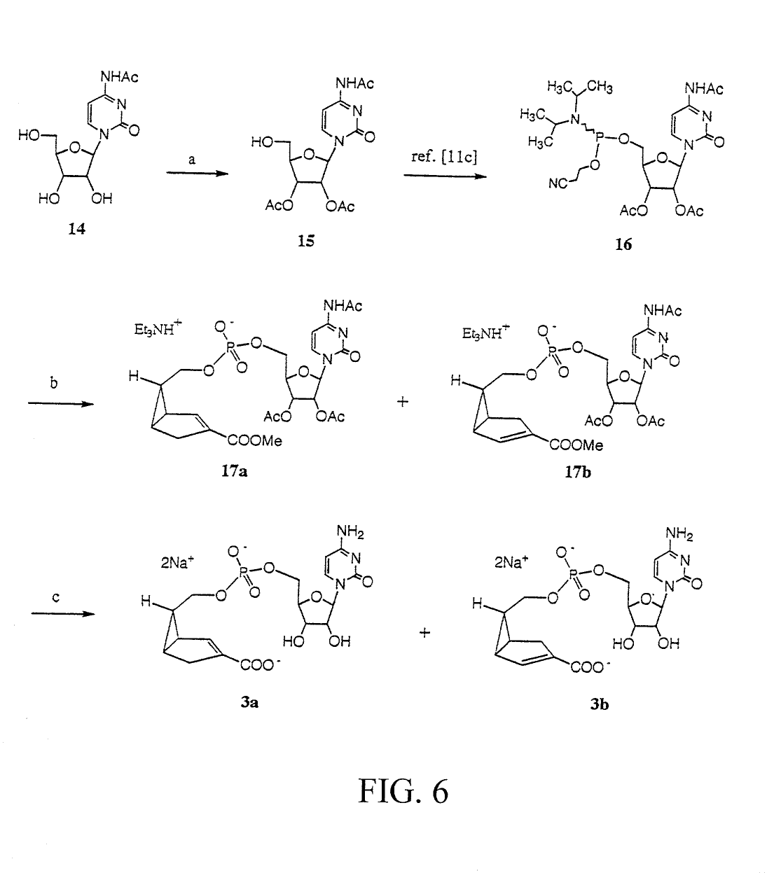

The subject invention provides methods of making glycosyltransferase inhibitors. In one embodiment, the bicyclic structure of compound 3a can be established by Meinwald rearrangement of norbornadiene 4. (Gilbert, J. C.; Smith, K. R. [1976] J. Org. Chem. 41, 3883.) An exemplary synthetic scheme is provided in Examples 1-2 and FIGS. 5-6.

In another embodiment, the subject invention provides for methods of suppressing, reducing, or inhibiting the enzymatic activity of one or more glycosytransferases or glycosylhydrolases by contacting the glycosytransferases with a composition containing an inhibitor of the instant invention and an amount sufficient to modulate the activity of the glycosytransferases. These methods may be practiced in vitro or in vivo.

The subject invention also provides methods of treating diseases or conditions in which inhibition, suppression, or reduction of glycosyltransferase or glycosylhydrolase activity provides for therapeutic benefit. In this embodiment of the invention, a subject in need of treatment is provided with a therapeutically effective amount of a pharmaceutical composition comprising one or more of the glycosyltransferase inhibitors of the invention. The compositions may be provided by, for example, injection, suppository, oral administration, and nasal administration.

Non-limiting examples of diseases or conditions suitable for treatment in accordance with the subject invention include those associated with bacterial, fungal, and viral infections, host-pathogen interaction, inflammation, tumor growth, tumor angiogenesis, tumor invasion and spread, malignant ascites, malignant pleural effusion, and metastasis. Other diseases and conditions suitable for treatment with compounds and compositions according to the invention include modulation of HIV infection, modulation of the immune response (e.g., hyperacute xenotransplant rejection or transplantation rejection), immunosupression, the development of dental plaque/caries, apoptosis, diseases associated with abnormal cellular adhesion patterns (such as scarring, keratinosis), intracellular communication and signal transduction pathways, and cellular development/differentiation.

It should be understood that the examples and embodiments described herein are for illustrative purposes only and that various modifications or changes in light thereof will be suggested to persons skilled in the art and are to be included within the spirit and purview of this application.

### EXAMPLE 1

Exemplary Synthetic Pathway for Glycosyltransferase Inhibitors

Meinwald rearrangement of norbornadiene 4 with m-chloroperoxybenzoic acid and subsequent reduction with sodium borohydride yielded racemic 6-endo-hydroxymethyl bicyclo[3.1.0]hex-2-ene. Protection using tert-butyldimethylsilyl chloride or tert-butyldiphenylsilyl chloride in the presence of imidazole proceeded in 85-90% yields.

Hydroboration-oxidation furnished the desired alcohol 5a and its regioisomer 11a in high yields (5a:11a≈5:2, by NMR spectroscopy). The ratio of diastereomers produced by hydroboration was insensitive to the O-protecting group. Stereoisomers 5a/11a or 5b/11b could be separated by careful column chromatography. Oxidation of 5a or 5b with chromium trioxide-pyridine gave ketone 6a (78%) or ketone 6b (86%), respectively. Alternatively, a mixture of 5b/11b was used for oxidation reaction to give the desired ketone 6b, which could be easily separated from its regioisomer by flash column chromatography.

Reaction of ketone 6b with trifluoromethanesulfonic anhydride using 2,6-di-tert-butyl-4-methylpyridine as a base afforded vinyl triflate 7 in 69% yield. Palladium-catalyzed carbonylation of 7 in methanol proceeded smoothly to form the unsaturated ester 8 in 80% yield. (Cacchi, S.; Morera, E.; Ortar, G. [1985] Tetrahedron Lett. 26, 1109.) Deprotection of 8 with tetrabutylammonium fluoride (TBAF) gave the key intermediate 9 in 85% yield.

Cytidine-2′,3′-di-O-acetyl-4-N-acetyl-5′-(N,N-diisopropyl-2-cyanoethyl)-phosphoramidite was prepared from the triacetyl cytidine (Zielinski, W. S.; Smrt, J.; Beranek, J. [1974] Coll. Czech. Chem. Commun. 39, 3560) by the literature method. (Kajihara, Y.; Ebata, T.; Koseki, K.; Kodama, H.; Matsushita, H.; Hashimoto, H. [1995] J. Org. Chem. 60, 5732.) We found that a more facile synthesis of the triacetyl cytidine could be carried out with the following one-pot sequence. Selective protection of the 5′-hydroxyl group of N-acetyl-cytidine (Watanabe, K. A.; Fox, J. J. [1966] Angew. Chem. Int. Ed. 6, 579) with 4,4′-dimethoxytrityl chloride in pyridine, followed by acetylation of the 2′,3′-hydroxyl groups with acetic anhydride in pyridine and deprotection of the 5′-hydroxyl group with 90% aqueous acetic acid, gave the required triacetate in 41% overall yield. The coupling of 9 with the cytidine 5′-phosphoramidite in the presence of tetrazole followed by oxidation with tert-butyl hydroperoxide and base-catalyzed removal of the cyanoethyl group gave a diastereomeric mixture of 10a and 10b.

Saponification of 10a/10b with sodium methoxide in methanol-water afforded a mixture of 3a and 3b (1:1 by NMR), which was purified by anion-exchange HPLC (NH₄HCO₃—MeOH—H₂O buffer as eluent).

Selected data for 9: ¹H NMR (300 MHz, CD₃Cl) δ 6.75(m, 1H), 3.71(s, 3H), 3.53(m, 1H), 3.36(m, 1H), 2.76(m, 1H), 2.45(m, 1H), 2.20(m, 1H), 1.89(m, 1H), 1.48(m, 1H), 1.38(bs, 1H); ¹³C NMR (75 MHz, CD₃Cl) δ 164.87, 141.61, 135.68, 56.31, 51.39, 31.14, 28.88, 24.10, 21.38; HRMS calculated for C₉H₁₃O₃ (M⁺+H): 169.0865; found: 169.090; for 3a/3b: ¹H NMR (300 MHz, D₂O) δ 7.75(d, J=7.62 Hz, 1H), 6.29(bs, 1H), 5.88(d, J=7.62 Hz, 1H), 5.75(m, 1H), 4.45(m, 3H), 3.93(m, 1H), 3.83(m, 1H), 3.58(m, 1H), 3.44(m, 1H), 2.45(m, 1H), 2.20˜2.18(m, 2H), 1.69(m, 1H), 1.28(m, 1H); ¹³C NMR (75 MHz, D₂O) δ 181.87, 173.91, 166.47, 158.12, 142.10, 141.61, 137.61, 96.82, 89.63, 89.52, 83.01, 74.46, 69.70, 64.46, 61.68, 31.96, 28.83, 23.65, 21.01; HRMS calculated for C₁₇H₂₁N₃O₁₀ P (M⁺−2Na⁺+H): 458.0965; found: 458.087.

### EXAMPLE 2

Exemplary Synthetic Pathway for Glycosyltransferase Inhibitors

Another route for the preparation of another important intermediate cytidine-phosphitamide 16 follows procedures set forth by Zielinski et al. ([1974] Coll. Czech. Chem. Commun. 39, 3560). A more facile synthesis of 16 can be carried out as depicted in Scheme 2. Selected protection of 5′-hydroxy group of N-Acetyl-cytidine 14 (Watanabe, K. A.; Fox, J. J. [1966] Angew. Chem. Int. Ed. 6, 579) with 4,4′-dimethoxytrityl chloride in pyridine, followed by acetylation of 2′,3′-hydroxy groups with acetic anhydride in pyridine and then deprotection of 5′-hydroxy group with 90% aqueous acetic acid, gave compound 15 in 41% overall yields in an one-pot reaction. Cytidine-phosphitamide 16 was prepared from 15 according to a literature procedure (Kajihara, Y.; Ebata, T.; Koseki, K.; Kodama, H.; Matsushita, H.; Hashimoto, H. [1995] J. Org. Chem. 60, 5732). Condensation of 11 and 16 in the presence of tetrazole followed by oxidation with tert-butyl hydroperoxide and base-catalyzed removal of the cyanoethyl group gave a diastereomeric mixture of 17a and 17b. Hydrolysis of 17a/17b with sodium methoxide in methanol-water afforded a mixture of 3a and 3b (1:1 by NMR), which was purified by HPLC (NH₄HCO₃—MeOH—H₂O buffer as eluent).

### EXAMPLE 3

Inhibitory Effects on Glycosytransferases

The inhibitory effects of 3a/3b on rat 2,3 and 2,6-sialytransferases were investigated with the use of radiolabeled [9-³H] CMP-NeuAc as a donor substrate. The acceptor sugars employed were lactose (for 2,3-sialytransferase) and lacNac (for 2,6-sialytransferase). The results show that 3a/3b are competitive inhibitors of both 2,3 and 2,6-sialyltransferases, and the Kᵢ's were estimated to be 10 μM and 20 μM for 2,3 and 2,6-sialytransferases, respectively.

### EXAMPLE 4

Uses, Formulations, and Administrations

Therapeutic and prophylactic application of the subject compounds, and compositions comprising them, can be accomplished by any suitable method and technique presently or prospectively known to those skilled in the art. Further, the compounds of the invention have use as starting materials or intermediates for the preparation of other useful compounds and compositions. Therefore, the compounds of the invention are useful for various non-therapeutic and therapeutic purposes.

The dosage administered will be dependent upon the response desired; the type of host involved; its age, health, weight, kind of concurrent treatment, if any; frequency of treatment; therapeutic ratio and like considerations. Advantageously, dosage levels of the administered active ingredients can be, for examples, dermal, 1 to about 500 mg/kg; orally, 0.01 to 200 mg/kg; intranasal 0.01 to about 100 mg/kg; and aerosol 0.01 to about 50 mg/kg of animal body weight.

Expressed in terms of concentration, the active ingredient of the invention can be present in the new compositions for use dermal, intra-nasally, bronchially, intramuscularly, intra-arterially, intra-vaginally, intra-venous, or orally in a concentration of from about 0.01 to about 50% w/w of the composition, and especially from about 0.1 to about 30% w/w of the composition. Preferably, the novel compound is present in a composition from about 1 to about 10%.

The compositions of the invention are advantageously used in a variety of forms (e.g., tablets, ointments, capsules, pills, powders, aerosols, granules, and oral solutions or suspensions and the like) containing the indicated suitable quantities of the active ingredient. Such compositions are referred to herein and in the accompanying claims generically as “pharmaceutical compositions.” Typically, they can be in unit dosage form, namely, in physically discrete units suitable as unitary dosages for human or animal subjects, each unit containing a predetermined quantity of active ingredient calculated to produce the desired therapeutic or prophylactic effect in association with one or more pharmaceutically acceptable other ingredients, e.g., diluent or carrier.

Where the pharmaceutical compositions are aerosols (e.g., for intra-nasal administration), the active ingredients can be packaged in pressurized aerosol containers with a propellant (e.g., carbon dioxide, nitrogen, propane, etc.) with the usual adjuvants such as cosolvents and/or wetting agents. Where the pharmaceutical compositions are ointments, the active ingredient can be mixed with a diluent vehicle such as cocoa butter, viscous polyethylene glycols, hydrogenated oils, and such mixtures can be emulsified if desired.

The compounds of the subject invention can also be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive compound(s) is combined with a suitable carrier in order to facilitate effective administration of the composition.

The isolated enantiomeric forms of the compounds of the invention are substantially free from one another (i.e., in enantiomeric excess). In other words, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. In one embodiment of the invention, the compounds are at least about in 90% enantiomeric excess. In a preferred embodiment, the compounds are in at least 95% enantiomeric excess. In a more preferred embodiment, the compounds are in at least 97.5% enantiomeric excess. In an even more preferred embodiment, the compounds are in at least 99% enantiomeric excess.

In one aspect, the subject invention provides pharmaceutical compositions comprising, as an active ingredient, an effective amount of one or more of the compounds and one or more non-toxic, pharmaceutically acceptable carriers or diluents. Examples of such carriers for use in the invention include ethanol, dimethyl sulfoxide, glycerol, silica, alumina, starch, talc, flour, and equivalent carriers and diluents.

Further, acceptable carriers can be either solid or liquid. Solid form preparations include powders, tablets, pills, capsules, cachets, suppositories and dispersible granules. A solid carrier can be one or more substances which may act as diluents, flavoring agents, solubilizers, lubricants, suspending agents, binders, preservatives, tablet disintegrating agents or an encapsulating material.

The disclosed pharmaceutical compositions may be subdivided into unit doses containing appropriate quantities of the active component. The unit dosage form can be a packaged preparation, such as packeted tablets, capsules, and powders in paper or plastic containers or in vials or ampules. Also, the unit dosage can be a liquid based preparation or formulated to be incorporated into solid food products, chewing gum, or lozenges.

1. A chemical compound comprising the formula: 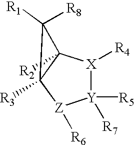
wherein R₁-R₈ are moieties selected from the group consisting of R₉, CH₃, alkyl groups, substituted alkyl groups, halogen, carboxyl, hydroxyl, phosphate, phosphonate, sugar residues, sugars, aryl, nucleosides, nucleoside monophosphates, nucleoside disphosphates, nucleoside triphosphates, and hydrogen;
R₉ is 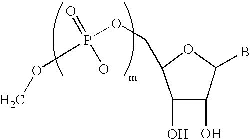
wherein B is adenine, thymine, guanine, cytosine, uracil, nicotinamide, or analogs thereof;
m is 1 or 2;
X, Y, and Z are carbon, nitrogen, oxygen, or sulfur and a double bond may, optionally, exist between atoms X and Y or atoms Y and Z; and
salts or isolated enantiomers of said chemical compound.

2. The chemical compound according to claim 1, wherein said substituted alkyl groups are substituted with a moiety selected from the group consisting Of C₁₋₆ alkyl, halogen, CN, OH, COOH, NO₂, NH₂, SO₂₋₄, C₁₋₂₀ heteroalkyl, C₂₋₂₀ alkenyl, alkynyl, akynyl-aryl, alkynyl-heteroaryl, aryl, C₁₋₂₀ alkyl-aryl, C₂₋₂₀ alkenyl-aryl, heteroaryl, C₁₋₂₀ alkyl-heteroaryl, C₂₋₂₀ alkenyl-heteroaryl, cycloalkyl, heterocycloalkyl, C₁₋₂₀ alkyl-heteroycloalkyl, and C₁₋₂₀ alkyl-cycloalkyl, any of which may be, optionally, substituted with a moiety selected from the group consisting of C₁₋₆ alkyl, halogen, OH, NH₂, CN, NO₂, COOH, or SO₂₋₄.

3. The chemical compound according to claim 1, wherein said salt is a hydrohloride, hydrobromide, p-toluenesulfonate, phosphate, sulfate, perchlorate, acetate, trifluororacetate, propionate, citrate, malonate, succinate, lactate, oxalate, tartrate, benzoate, magnesium, calcium, morpholine, piperidine, dimethylamine, or diethylamine salt.

4. The chemical compound according to claim 1, wherein said isolated enantiomeric forms of the chemical compound are substantially free from one another.

5. The chemical compound according to claim 4, wherein said isolated enantiomeric forms of said chemical compound is at least about in 90%, 95%, 97.5%, or 99% enantiomeric excess.

6. A composition comprising a carrier and a chemical compound comprising the formula: 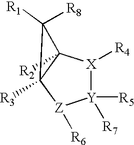
wherein R₁-R₈ are moieties selected from the group consisting of R₉, CH₃, alkyl groups, substituted alkyl groups, halogen, carboxyl, hydroxyl, phosphate, phosphonate, sugar residues, sugars, aryl, nucleosides, nucleoside monophosphates, nucleoside disphosphates, nucleoside triphosphates, and hydrogen;
R₉ is 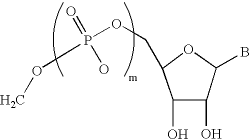
wherein B is adenine, thymine, guanine, cytosine, uracil, nicotinamide, or analogs thereof;
m is 1 or 2;
X, Y, and Z are carbon, nitrogen, oxygen, or sulfur and a double bond may, optionally, exist between atoms X and Y or atoms Y and Z; and
salts or isolated enantiomers of said chemical compound.

7. The composition according to claim 6, wherein said substituted alkyl groups are substituted with a moiety selected from the group consisting of C₁₋₆ alkyl, halogen, CN, OH, COOH, NO₂, NH₂, SO₂₋₄, C₁₋₂₀ heteroalkyl, C₂₋₂₀ alkenyl, alkynyl, akynyl-aryl, alkynyl-heteroaryl, aryl, C-₁₋₂₀ alkyl-aryl, C₂₋₂₀ alkenyl-aryl, heteroaryl, C₁₋₂₀ alkyl-heteroaryl, C₂₋₂₀ alkenyl-heteroaryl, cycloalkyl, heterocycloalkyl, C₁₋₂₀ alkyl-heteroycloalkyl, and C₁₋₂₀ alkyl-cycloalkyl, any of which may be, optionally, substituted with a moiety selected from the group consisting of C₁₋₆ alkyl, halogen, OH, NH₂, CN, NO₂, COOH, or SO₂₋₄.

8. The composition according to claim 6, wherein said carrier is a pharmaceutical carrier.

9. The composition according to claim 8, wherein said pharmaceutical carrier is solid, liquid, or aerosol.

10. The composition according to claim 6, wherein said composition is in unit dose form.

11. The composition according to claim 6, wherein said substituted alkyl groups are substituted with a moiety selected from the group consisting of C₁₋₆ alkyl, halogen, CN, OH, COOH, NO₂, NH₂, SO₂₋₄, C₁₋₂₀ heteroalkyl, C₂₋₂₀ alkenyl, alkynyl, akynyl-aryl, alkynyl-heteroaryl, aryl, C₁₋₂₀ alkyl-aryl, C₂₋₂₀ alkenyl-aryl, heteroaryl, C₁₋₂₀ alkyl-heteroaryl, C₂₋₂₀ alkenyl-heteroaryl, cycloalkyl, heterocycloalkyl, C₁₋₂₀ alkyl-heteroycloalkyl, and C ₁₋₂₀ alkyl-cycloalkyl, any of which may be, optionally, substituted with a moiety selected from the group consisting of C₁₋₆ alkyl, halogen, OH, NH₂, CN, NO₂, COOH, or SO₂₋₄.

12. The composition according to claim 6, wherein said salt is a hydrohloride, hydrobromide, p-toluenesulfonate, phosphate, sulfate, perchlorate, acetate, trifluororacetate, propionate, citrate, malonate, succinate, lactate, oxalate, tartrate, benzoate, magnesium, calcium, morpholine, piperidine, dimethylamine, or diethylamine salt.

13. The composition according to claim 6, wherein said isolated enantiomeric forms of the chemical compound are substantially free from one another.

14. The composition according to claim 6, wherein said isolated enantiomeric forms of said compound is at least about in 90%, 95%, 97.5%, or 99% enantiomeric excess.

15. The composition according to claim 6, wherein said carrier is a powder, tablet, pill, capsule, cachet, suppository, or dispersible granule.

16. A method of suppressing, reducing, or inhibiting glycosyltransferase or glycosylhydrolase activity comprising contacting said glycosyltransferase or glycosylhydrolase with a composition, in an amount sufficient to suppress, reduce, or inhibit said glycosyltransferase or glycosyltransferase activity, comprising a carrier and a chemical compound comprising the formula: 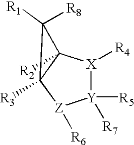
wherein R₁-R₈ are moieties selected from the group consisting of R₉, CH₃, alkyl groups, substituted alkyl groups, halogen, carboxyl, hydroxyl, phosphate, phosphonate, sugar residues, sugars, aryl, nucleosides, nucleoside monophosphates, nucleoside disphosphates, nucleoside triphosphates, and hydrogen;
R₉ is 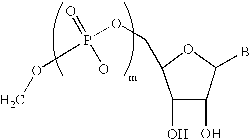
wherein B is adenine, thymine, guanine, cytosine, uracil, nicotinamide, or analogs thereof,
m is 1 or 2;
X, Y, and Z are carbon, nitrogen, oxygen, or sulfur and a double bond may, optionally, exist between atoms X and Y or atoms Y and Z; and
salts or isolated enantiomers of said chemical compound.

17. The method according to claim 16, wherein said composition comprises a hydrochloride, hydrobromide, p-toluenesulfonate, phosphate, sulfate, perchlorate, acetate, trifluororacetate, propionate, citrate, malonate, succinate, lactate, oxalate, tartrate, benzoate, magnesium, calcium, morpholine, piperidine, dimethylamine, or diethylamine salt of said chemical compound.

18. The method according to claim 16, wherein said composition comprises isolated enantiomeric forms of said chemical compounds.

19. The method according to claim 18, wherein said isolated enantiomeric forms of said compound is in at least about 90%, 95%, 97.5%, or 99% enantiomeric excess.

20. The method according to claim 16, wherein said suppression, reduction, or inhibition of said glycosyltransferase or glycosylhydrolase activity provides therapeutic benefit.
